SEQUENCE LISTING (26 sequences)

SEQ ID NO: 1 - protein, 6 aa, Artificial Sequence
  Synthetic construct
RLVLAS

SEQ ID NO: 2 - protein, 7 aa, Artificial Sequence
  Synthetic construct
CRLVLAS

SEQ ID NO: 3 - protein, 7 aa, Artificial Sequence
  Synthetic construct
RLVLASC

SEQ ID NO: 4 - protein, 12 aa, Artificial Sequence
  Synthetic construct
CYGRKKRRQRRR

SEQ ID NO: 5 - protein, 12 aa, Artificial Sequence
  Synthetic construct
YGRKKRRQRRRC

SEQ ID NO: 6 - protein, 7 aa, Artificial Sequence
  Synthetic construct
XRLVLAS

SEQ ID NO: 7 - protein, 7 aa, Artificial Sequence
  Synthetic construct
RLVLASX

SEQ ID NO: 8 - protein, 12 aa, Artificial Sequence
  Synthetic construct
XYGRKKRRQRRR

SEQ ID NO: 9 - protein, 12 aa, Artificial Sequence
  Synthetic construct
YGRKKRRQRRRX

SEQ ID NO: 10 - protein, 7 aa, Artificial Sequence
  Synthetic construct
XRLVLAS

SEQ ID NO: 11 - protein, 12 aa, Artificial Sequence
  Synthetic construct
XYGRKKRRQRRR

SEQ ID NO: 12 - protein, 12 aa, Artificial Sequence
  Synthetic construct
XYGRKKRRQRRR

SEQ ID NO: 13 - protein, 8 aa, Artificial Sequence
  Synthetic construct
ACRLVLAS

SEQ ID NO: 14 - protein, 13 aa, Artificial Sequence
  Synthetic construct
ACYGRKKRRQRRR

SEQ ID NO: 15 - protein, 8 aa, Artificial Sequence
  Synthetic construct
RLVLASGG

SEQ ID NO: 16 - protein, 9 aa, Artificial Sequence
  Synthetic construct
RLVASGGKC

SEQ ID NO: 17 - protein, 9 aa, Artificial Sequence
  Synthetic construct
RLVASGGKX

SEQ ID NO: 18 - protein, 19 aa, Artificial Sequence
  Synthetic construct
RLVLASGGYGRKKRRQRRR

SEQ ID NO: 19 - protein, 19 aa, Artificial Sequence
  Synthetic construct
YGRKKRRQRRRGGRLVLAS

SEQ ID NO: 20 - protein, 19 aa, Artificial Sequence
  Synthetic construct
RLVASGGKYGRKKRRQRRR

SEQ ID NO: 21 - protein, 19 aa, Artificial Sequence
  Synthetic construct
SALVLRGGRRRQRRKKRGY

SEQ ID NO: 22 - protein, 19 aa, Artificial Sequence
  Synthetic construct
SALVLRGGYGRKKRRQRRR

SEQ ID NO: 23 - protein, 7 aa, Artificial Sequence
  Synthetic construct
XRLVLAS

SEQ ID NO: 24 - protein, 7 aa, Artificial Sequence
  Synthetic construct
XRLVLAS

SEQ ID NO: 25 - protein, 12 aa, Artificial Sequence
  Synthetic construct
XYGRKKRRQRRR

SEQ ID NO: 26 - protein, 11 aa, Artificial Sequence
  Synthetic construct
YGRKKRRQRRR

(19) United States
(12) Patent Application Publication
(10) Pub. No.: US 2009/0062178 A1
(43) Pub. Date: Mar. 5, 2009

(54) METHODS OF USE OF GAMMA INHIBITOR COMPOUNDS FOR THE ATTENUATION OF PAIN

(76) Inventor: Stephen D. HARRISON, Albany, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/099,074

(22) Filed: Apr. 7, 2008

Related U.S. Application Data
(60) Provisional application No. 60/910,588, filed on Apr. 6, 2007.

(51) Int. Cl. C07K 7/06; C07K 2/00; C07K 7/08; A61K 38/02; A61P 29/00

(52) U.S. Cl. 514/2; 530/300; 530/327; 530/329; 530/328

(57) ABSTRACT

The disclosure herein relates to modified γPKC inhibitory peptides, methods of generating such peptides, and method for using γPKC inhibitory peptides for the treatment of pain.

### CROSS-REFERENCE TO RELATED APPLICATIONS

This application claims priority from U.S. provisional application 60/910,588 filed Apr. 6, 2007. The contents of this document are incorporated by reference in its entirety.

### TECHNICAL FIELD

The present disclosure relates to compounds that modulate different categories of pain, wherein the compounds comprise one or more gamma PKC (γPKC) inhibitory peptides coupled to at least one carrier moiety and where the inhibitory peptides, the carrier moiety, or both have been modified from a prototype sequence to increase the stability, potency, or both of the resulting compound.

### BACKGROUND ART

Protein kinase C (“PKC”) is a key enzyme in signal transduction involved in a variety of cellular functions, including cell growth, regulation of gene expression, and ion channel activity. The PKC family of isozymes includes at least 11 different protein kinases that can be divided into at least three subfamilies based on their homology and sensitivity to activators. The families are the classical, the novel, and the atypical subfamilies. Each isozyme includes a number of homologous (“conserved” or “C”) domains interspersed with isozyme-unique (“variable” or “V”) domains. Gamma PKC (γPKC) is a member of the “conventional” subfamily, along with α, β_{I} (also known as B₂), and β_{II} {also known as B₁)) PKC.

Individual isozymes of PKC have been implicated in the mechanisms of various disease states. Epsilon PKC inhibitory peptides derived from εPKC have been generated and shown to impact nociception. For example, see U.S. Pat. Nos. 6,376,467 and 6,686,334. Gamma PKC inhibitory peptides derived for γPKC have also been enclosed U.S. Publication No. 20030223981, which is hereby incorporated by reference.

One problem with this approach is that the “naked” termini of the excised fragments are different from their context in the protein, revealing free amine and carboxyl groups at the points where the fragment attaches to the remainder of the protein. These extraneous moieties may render the peptide more susceptible to proteases. As a result of these liabilities the potency of the peptide may be less than desired and the in vivo half-life may be significantly shortened.

A second area of the prior art makes use of a similar strategy, wherein “carrier” peptides are designed as fragments of HIV-Tat and other proteins. These peptide fragments mimic the ability of the parent protein to cross cell membranes. Of particular interest is the property that “cargo” peptides can be attached to these carrier peptides such that both cargo and carrier peptides are carried into the cell by these carrier peptide fragments.

Recognizing that the carrier peptides are fragments, similar deficiencies may apply as noted above for the cargo peptides. That is, the exposed termini may confer undesirable properties including protease susceptibility.

Prior art cargo/carrier peptide constructs have made use of a Cys-Cys disulfide bond between cargo and carrier, which can be cleaved by a number of agents, such as glutathione reduction when the peptides enter cells. This property has been thought to be important for biological activity, since the physical separation of cargo and carrier allows the two moieties to exert their independent effects within the cell. However, this hypothesis has not been convincingly tested, and non-cleavable analogs may in fact have good activity. Further, the disulfide bond is cumbersome to assemble, and prone to chemical degradation.

The design of certain prior art cargo/carrier peptides is based on a contiguous sequence of amino acids from the protein. However, the optimal length of the peptide has not yet been well defined, being based on sequence comparison analysis and theoretical prediction of the desired sequence rather than on an empirical basis of analog testing. Thus, increased potency may be anticipated from analogs of the previously described cargo peptides which contain additional residues corresponding to the γPKC domain from which the have been derived.

### BRIEF DESCRIPTION OF THE DRAWINGS

FIG. 1 shows a Western blot of samples treated with a γPKC inhibitory protein showing the impact of the inhibitor on enzyme levels in the cytosol and on membrane fractions.

FIG. 2 shows a line graph plotting the number of paw withdrawals against days post-L5 transection in a study using a 2 gram Von Frey filament.

FIG. 3 shows a line graph plotting the number of paw withdrawals against days post-L5 transection in a study using a 12 gram Von Frey filament.

FIGS. 4A and 4B show two line graphs plotting the averaged number of paw withdrawals against days post-transection and a crossover event at day 7.5 post transection in two studies using a 2 and a 12 gram Von Frey filament.

FIG. 5 shows a line graph plotting paw withdrawal latency in seconds against days post-L5 transection in a study of thermal hyperalgesia.

FIG. 6 shows a line graph plotting paw withdrawal latency in seconds against days post-L5 transection in a study of thermal hyperalgesia with a crossover event at day 7.5.

FIG. 7 shows a line graph plotting paw withdrawal latency in seconds against time in a study of thermal hyperalgesia where animals were challenged with a dose of inhibitory peptide administered subcutaneously on day 14 after receiving the peptide via pump for days 1-7 post transection.

FIG. 8 shows a line graph plotting paw withdrawal latency in seconds against time in a study of thermal hyperalgesia where animals were challenged with a dose of inhibitory peptide administered subcutaneously on day 14 after receiving the peptide via pump for days 7-14 post transection.

FIG. 9 shows a line graph plotting paw withdrawal latency in seconds against time in a study of thermal hyperalgesia where animals were challenged with a dose of inhibitory peptide administered subcutaneously on day 14 post transection.

### DISCLOSURE OF THE INVENTION

The disclosure herein relates to modified γPKC inhibitory peptides, methods of generating such peptides, and method for using γPKC inhibitory peptides for the treatment of pain. Other aspects and embodiments will be apparent to those skilled in the art form the following detailed description.

### DESCRIPTION OF THE INVENTION

The presently described invention relates to modified peptides which inhibit the gamma protein kinase C (γPKC) isozyme. Typically, the γPKC inhibitory peptides discussed herein are coupled to a carrier moiety to facilitate transport of the inhibitory peptide to a target cell. The cargo inhibitory peptide, the carrier peptide, or both can be modified relative to a prototype control to increase the stability of the resulting cargo/carrier peptide constructs. The disclosed modified γPKC peptides are useful in preventing and treating various types of pain, such as acute pain, chronic pain, and inflammatory pain.

### DEFINITIONS

As used in the present specification, the following words and phrases are generally intended to have the meanings as set forth below, except to the extent that the context in which they are used indicates otherwise.

A “PKC inhibitory peptide” refers to a peptide that can inhibit or inactivate an γPKC enzyme.

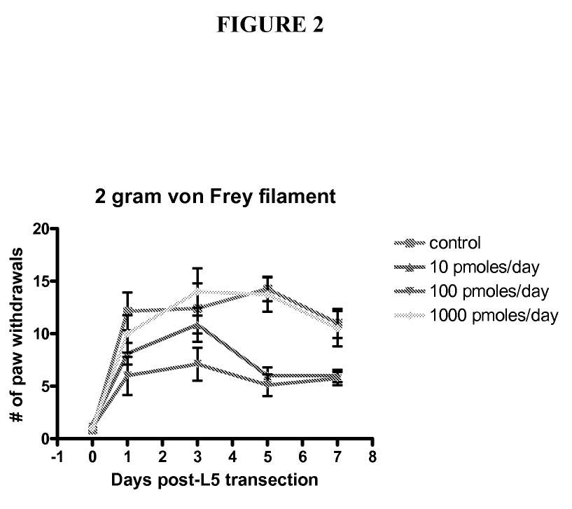

The term “capped” refers to a peptide that has been chemically modified to alter the amino terminus, carboxy terminus, or both. A capped carrier peptide disulfide bonded to an unmodified cargo peptide is shown in FIG. 2.

The term “carrier” refers to a moiety that facilitates cellular uptake, such as cationic polymers, peptides and antibody sequences, including polylysine, polyarginine, Antennapedia-derived peptides, HIV Tat-derived peptides and the like, as described, for example, in U.S. Pat. Nos. and Publications Nos. 4,847,240, 5,888,762, 5,747,641, 6,593,292, US2003/0104622, US2003/0199677 and US2003/0206900. An example of a carrier moiety is a “carrier peptide,” which is a peptide which facilitates cellular uptake of a γPKC inhibitory peptide which is chemically associated or bonded to the transporter peptide.

The term “prophylaxis” is intended as an element of “treatment” to encompass both “preventing” and “suppressing” as defined herein. It will be understood by those skilled in the art that in human medicine it is not always possible to distinguish between “preventing” and “suppressing” since the ultimate inductive event or events may be unknown, latent, or the patient is not ascertained until well after the occurrence of the event or events.

The term “stability” refers generally to modifications that improve shelf-life times, for example, retarding shelf life-based cys-cys exchange, by retarding proteolytic degradation, or both. The term “potency” relates to the amount of a particular peptide composition required to achieve a particular result. One peptide composition is more potent than another when dosages of the composition can be reduced to achieve a desired end point. Certain modifications of a given peptide composition can be made with improve potency of that composition.

Gamma Protein Kinase C (γPKC) Inhibitory Peptides

Various γPKC inhibitors are described herein and can be used with the presently disclosed methods. The inhibitory peptide can be derived from any domain, whether variable or constant. Thus, inhibitory peptides can be derived from V1, V2, V3, V4, or V5. Inhibitory peptides can also be derived from the constant regions C1 (C1a, C1b), C3, C4, or C5. Peptides overlapping one or more of these regions are also contemplated. The cargo peptides derived from the various domains and range in length from 5 to 30 amino acids in length. More particularly, the peptides derived from the PKC domain are 5, 6, 7, 8, 9, 10, 11, 12, 13, 14, 15, 16, 17, 18, 19, 20, 21, 22, 23, 24, 25, 26, 27, 28, 29, or 30 residues in length. Another source of prototype peptides can be found in U.S. patent application Ser. No. 11/011,557, entitled, “Isozyme-specific antagonists of protein kinase C,” which takes activator peptides and converts them to inhibitor peptides, and which is hereby incorporated by reference in its entirety.

In one embodiment, the cargo peptide is an γPKC inhibitory peptide derivative of γPKC comprising the amino acid sequence of R-L-V-L-A-S (SEQ ID NO:1), a cysteine residue located at the amino or carboxy terminal ends of the peptide, or internally, and a carrier peptide linked to the cargo peptide. The cargo peptide described above can further comprise one or more additional cargo peptides, attached to one another and ultimately to the carrier peptide.

Modifications to both the carrier and cargo have been made with the goals of improving potency, stability in biological fluids/tissues, and chemical stability. These changes provide a γPKC inhibitor with enhanced properties for use in a variety of clinical indications.

Some of the modifications which have been applied include:
1. Capping the cargo and/or carrier peptides to hinder proteolysis in vivo, and thereby to increase potency and/or duration of efficacy;
2. Generating overlap peptides incorporating additional contiguous regions of the parent protein to improve potency;
3. Making linear peptides which have cargo and carrier in a single peptide chain to improve the chemical stability and shelf-life of drug product;
4. Making multimer peptides which have two or more copies of the active peptide to improve protease resistance and potency;
5. Making retro-inverso analogs of peptides to hinder proteolysis; and
6. Introducing disulfide analogs to provide improved chemical stability.

The modifications described herein improve the potency, plasma stability, and chemical stability of the modified γPKC inhibitory peptides. Effective modifications to γPKC inhibitory peptides are identified by selecting a prototype γPKC inhibitory peptide and modifying these peptides to serve as cargo peptides for the treatment of pain. The prototype peptide can be a presently known peptide or one as of yet unidentified as a γPKC inhibitory peptide. A preferred prototype sequence is R-L-V-L-A-S (SEQ ID NO:1), where the peptide is unmodified and conjugated to a carrier via Cys residues located at the amino termini of the cargo and carrier peptides, although any inhibitory γPKC peptide can be used as the starting cargo sequence. A variety of modified or analog peptides are contemplated. Some such analogs comprise amino acid sequences that overlap and extend beyond the prototype sequence. Other analog peptides are truncated relative to the prototype. Additionally, analogs of the prototype sequence may have one or more amino acid substitutions relative to the prototype sequence, wherein the amino acid substituted is an alanine residue or an aspartic acid residue. The systematic generation of such alanine or aspartic acid containing peptides is known as “scanning.” The generation of linear peptides comprising the analogs and modified carrier peptides is further contemplated.

Additional modifications to prototype sequences are directed at modifying specific degradation sites within the cargo peptide or peptides, the carrier peptide or peptides, or both, and introducing amino acid substitutions or other chemical modifications which blocks these sites from degradation.

The following tables list a number of exemplary gamma PKC inhibitory peptides for use with the present invention as prototype sequences.

TABLE 1

BASIC SET

| | | |
|---|---|---|
| CARGO | LINKER | CARRIER |

| | | | | | | |
|---|---|---|---|---|---|---|
| N-term | Cargo | C-term | Linker | N-term | Carrier | C-term |

| | | | | | | |
|---|---|---|---|---|---|---|
| Amine | CRLVLAS | Carboxyl | Disulfide | Amine | CYGRKKRRQRRR | Carboxyl |
| | SEQ ID | | | | SEQ ID NO: 4 | |
| | NO: 2 | | | | | |
| Acetyl | CRLVLAS | Carboxyl | Disulfide | Amine | CYGRKKRRQRRR | Carboxyl |
| Acetyl | CRLVLAS | Amide | Disulfide | Amine | CYGRKKRRQRRR | Carboxyl |
| Amine | CRLVLAS | Amide | Disulfide | Amine | CYGRKKRRQRRR | Carboxyl |
| Amine | CRLVLAS | Carboxyl | Disulfide | Acetyl | CYGRKKRRQRRR | Carboxyl |
| Amine | CRLVLAS | Carboxyl | Disulfide | Acetyl | CYGRKKRRQRRR | Amide |
| Amine | CRLVLAS | Carboxyl | Disulfide | Amine | CYGRKKRRQRRR | Amide |
| Acetyl | CRLVLAS | Amide | Disulfide | Acetyl | CYGRKKRRQRRR | Amide |
| Amine | RLVLASC | Carboxyl | Disulfide | Amine | CYGRKKRRQRRR | Carboxyl |
| | SEQ ID | | | | | |
| | NO: 3 | | | | | |
| Acetyl | RLVLASC | Carboxyl | Disulfide | Amine | CYGRKKRRQRRR | Carboxyl |
| Acetyl | RLVLASC | Amide | Disulfide | Amine | CYGRKKRRQRRR | Carboxyl |
| Amine | RLVLASC | Amide | Disulfide | Amine | CYGRKKRRQRRR | Carboxyl |
| Amine | RLVLASC | Carboxyl | Disulfide | Acetyl | CYGRKKRRQRRR | Carboxyl |
| Amine | RLVLASC | Carboxyl | Disulfide | Acetyl | CYGRKKRRQRRR | Amide |
| Amine | RLVLASC | Carboxyl | Disulfide | Amine | CYGRKKRRQRRR | Amide |
| Acetyl | RLVLASC | Amide | Disulfide | Acetyl | CYGRKKRRQRRR | Amide |
| Amine | RLVLASC | Carboxyl | Disulfide | Amine | YGRKKRRQRRRC | Carboxyl |
| | | | | | SEQ ID NO: 5 | |
| Acetyl | RLVLASC | Carboxyl | Disulfide | Amine | YGRKKRRQRRRC | Carboxyl |
| Acetyl | RLVLASC | Amide | Disulfide | Amine | YGRKKRRQRRRC | Carboxyl |
| Amine | RLVLASC | Amide | Disulfide | Amine | YGRKKRRQRRRC | Carboxyl |
| Amine | RLVLASC | Carboxyl | Disulfide | Acetyl | YGRKKRRQRRRC | Carboxyl |
| Amine | RLVLASC | Carboxyl | Disulfide | Acetyl | YGRKKRRQRRRC | Amide |
| Amine | RLVLASC | Carboxyl | Disulfide | Amine | YGRKKRRQRRRC | Amide |
| Acetyl | RLVLASC | Amide | Disulfide | Acetyl | YGRKKRRQRRRC | Amide |
| Amine | CRLVLAS | Carboxyl | Disulfide | Amine | YGRKKRRQRRRC | Carboxyl |
| Acetyl | CRLVLAS | Carboxyl | Disulfide | Amine | YGRKKRRQRRRC | Carboxyl |
| Acetyl | CRLVLAS | Amide | Disulfide | Amine | YGRKKRRQRRRC | Carboxyl |
| Amine | CRLVLAS | Amide | Disulfide | Amine | YGRKKRRQRRRC | Carboxyl |
| Amine | CRLVLAS | Carboxyl | Disulfide | Acetyl | YGRKKRRQRRRC | Carboxyl |
| Amine | CRLVLAS | Carboxyl | Disulfide | Acetyl | YGRKKRRQRRRC | Amide |
| Amine | CRLVLAS | Carboxyl | Disulfide | Amine | YGRKKRRQRRRC | Amide |
| Acetyl | CRLVLAS | Amide | Disulfide | Acetyl | YGRKKRRQRRRC | Amide |

TABLE 2

HOMOCYSTEINE (homoC)

| | | |
|---|---|---|
| CARGO | LINKER | CARRIER |

| | | | | | | |
|---|---|---|---|---|---|---|
| N-term | Cargo | C-term | Linker | N-term | Carrier | C-term |

| | | | | | | |
|---|---|---|---|---|---|---|
| Amine | homoC-RLVLAS | Carboxyl | Disulfide | Amine | homoC-YGRKKRRQRRR | Carboxyl |
| | SEQ ID NO: 6 | | | | SEQ ID NO: 8 | |
| Acetyl | homoC-RLVLAS | Carboxyl | Disulfide | Amine | homoC-YGRKKRRQRRR | Carboxyl |
| Acetyl | homoC-RLVLAS | Amide | Disulfide | Amine | homoC-YGRKKRRQRRR | Carboxyl |
| Amine | homoC-RLVLAS | Amide | Disulfide | Amine | homoC-YGRKKRRQRRR | Carboxyl |
| Amine | homoC-RLVLAS | Carboxyl | Disulfide | Acetyl | homoC-YGRKKRRQRRR | Carboxyl |
| Amine | homoC-RLVLAS | Carboxyl | Disulfide | Acetyl | homoC-YGRKKRRQRRR | Amide |
| Amine | homoC-RLVLAS | Carboxyl | Disulfide | Amine | homoC-YGRKKRRQRRR | Amide |
| Acetyl | homoC-RLVLAS | Amide | Disulfide | Acetyl | homoC-YGRKKRRQRRR | Amide |
| Amine | RLVLAS-homoC | Carboxyl | Disulfide | Amine | homoC-YGRKKRRQRRR | Carboxyl |
| | SEQ ID NO: 7 | | | | | |
| Acetyl | RLVLAS-homoC | Carboxyl | Disulfide | Amine | homoC-YGRKKRRQRRR | Carboxyl |
| Acetyl | RLVLAS-homoC | Amide | Disulfide | Amine | homoC-YGRKKRRQRRR | Carboxyl |
| Amine | RLVLAS-homoC | Amide | Disulfide | Amine | homoC-YGRKKRRQRRR | Carboxyl |
| Amine | RLVLAS-homoC | Carboxyl | Disulfide | Acetyl | homoC-YGRKKRRQRRR | Carboxyl |
| Amine | RLVLAS-homoC | Carboxyl | Disulfide | Acetyl | homoC-YGRKKRRQRRR | Amide |
| Amine | RLVLAS-homoC | Carboxyl | Disulfide | Amine | homoC-YGRKKRRQRRR | Amide |
| Acetyl | RLVLAS-homoC | Amide | Disulfide | Acetyl | homoC-YGRKKRRQRRR | Amide |
| Amine | RLVLAS-homoC | Carboxyl | Disulfide | Amine | YGRKKRRQRRR-homoC | Carboxyl |
| | | | | | SEQ ID NO: 9 | |
| Acetyl | RLVLAS-homoC | Carboxyl | Disulfide | Amine | YGRKKRRQRRR-homoC | Carboxyl |
| Acetyl | RLVLAS-homoC | Amide | Disulfide | Amine | YGRKKRRQRRR-homoC | Carboxyl |
| Amine | RLVLAS-homoC | Amide | Disulfide | Amine | YGRKKRRQRRR-homoC | Carboxyl |
| Amine | RLVLAS-homoC | Carboxyl | Disulfide | Acetyl | YGRKKRRQRRR-homoC | Carboxyl |
| Amine | RLVLAS-homoC | Carboxyl | Disulfide | Acetyl | YGRKKRRQRRR-homoC | Amide |
| Amine | RLVLAS-homoC | Carboxyl | Disulfide | Amine | YGRKKRRQRRR-homoC | Amide |
| Acetyl | RLVLAS-homoC | Amide | Disulfide | Acetyl | YGRKKRRQRRR-homoC | Amide |
| Amine | homoC-RLVLAS | Carboxyl | Disulfide | Amine | YGRKKRRQRRR-homoC | Carboxyl |
| Acetyl | homoC-RLVLAS | Carboxyl | Disulfide | Amine | YGRKKRRQRRR-homoC | Carboxyl |
| Acetyl | homoC-RLVLAS | Amide | Disulfide | Amine | YGRKKRRQRRR-homoC | Carboxyl |
| Amine | homoC-RLVLAS | Amide | Disulfide | Amine | YGRKKRRQRRR-homoC | Carboxyl |
| Amine | homoC-RLVLAS | Carboxyl | Disulfide | Acetyl | YGRKKRRQRRR-homoC | Carboxyl |
| Amine | homoC-RLVLAS | Carboxyl | Disulfide | Acetyl | YGRKKRRQRRR-homoC | Amide |
| Amine | homoC-RLVLAS | Carboxyl | Disulfide | Amine | YGRKKRRQRRR-homoC | Amide |
| Acetyl | homoC-RLVLAS | Amide | Disulfide | Acetyl | YGRKKRRQRRR-homoC | Amide |

TABLE 3

HOMOCYSTEINE (homoC) - Cargo only

| | | |
|---|---|---|
| CARGO | LINKER | CARRIER |

| | | | | | | |
|---|---|---|---|---|---|---|
| N-term | Cargo | C-term | Linker | N-term | Carrier | C-term |

| | | | | | | |
|---|---|---|---|---|---|---|
| Amine | homoC-RLVLAS | Carboxyl | Disulfide | Amine | CYGRKKRRQRRR | Carboxyl |
| Acetyl | homoC-RLVLAS | Carboxyl | Disulfide | Amine | CYGRKKRRQRRR | Carboxyl |
| Acetyl | homoC-RLVLAS | Amide | Disulfide | Amine | CYGRKKRRQRRR | Carboxyl |
| Amine | homoC-RLVLAS | Amide | Disulfide | Amine | CYGRKKRRQRRR | Carboxyl |
| Amine | homoC-RLVLAS | Carboxyl | Disulfide | Acetyl | CYGRKKRRQRRR | Carboxyl |
| Amine | homoC-RLVLAS | Carboxyl | Disulfide | Acetyl | CYGRKKRRQRRR | Amide |
| Amine | homoC-RLVLAS | Carboxyl | Disulfide | Amine | CYGRKKRRQRRR | Amide |
| Acetyl | homoC-RLVLAS | Amide | Disulfide | Acetyl | CYGRKKRRQRRR | Amide |
| Amine | RLVLAS-homoC | Carboxyl | Disulfide | Amine | CYGRKKRRQRRR | Carboxyl |
| Acetyl | RLVLAS-homoC | Carboxyl | Disulfide | Amine | CYGRKKRRQRRR | Carboxyl |
| Acetyl | RLVLAS-homoC | Amide | Disulfide | Amine | CYGRKKRRQRRR | Carboxyl |
| Amine | RLVLAS-homoC | Amide | Disulfide | Amine | CYGRKKRRQRRR | Carboxyl |
| Amine | RLVLAS-homoC | Carboxyl | Disulfide | Acetyl | CYGRKKRRQRRR | Carboxyl |
| Amine | RLVLAS-homoC | Carboxyl | Disulfide | Acetyl | CYGRKKRRQRRR | Amide |
| Amine | RLVLAS-homoC | Carboxyl | Disulfide | Amine | CYGRKKRRQRRR | Amide |
| Acetyl | RLVLAS-homoC | Amide | Disulfide | Acetyl | CYGRKKRRQRRR | Amide |
| Amine | RLVLAS-homoC | Carboxyl | Disulfide | Amine | YGRKKRRQRRRC | Carboxyl |
| Acetyl | RLVLAS-homoC | Carboxyl | Disulfide | Amine | YGRKKRRQRRRC | Carboxyl |
| Acetyl | RLVLAS-homoC | Amide | Disulfide | Amine | YGRKKRRQRRRC | Carboxyl |
| Amine | RLVLAS-homoC | Amide | Disulfide | Amine | YGRKKRRQRRRC | Carboxyl |
| Amine | RLVLAS-homoC | Carboxyl | Disulfide | Acetyl | YGRKKRRQRRRC | Carboxyl |
| Amine | RLVLAS-homoC | Carboxyl | Disulfide | Acetyl | YGRKKRRQRRRC | Amide |
| Amine | RLVLAS-homoC | Carboxyl | Disulfide | Amine | YGRKKRRQRRRC | Amide |
| Acetyl | RLVLAS-homoC | Amide | Disulfide | Acetyl | YGRKKRRQRRRC | Amide |
| Amine | homoC-RLVLAS | Carboxyl | Disulfide | Amine | YGRKKRRQRRRC | Carboxyl |
| Acetyl | homoC-RLVLAS | Carboxyl | Disulfide | Amine | YGRKKRRQRRRC | Carboxyl |
| Acetyl | homoC-RLVLAS | Amide | Disulfide | Amine | YGRKKRRQRRRC | Carboxyl |
| Amine | homoC-RLVLAS | Amide | Disulfide | Amine | YGRKKRRQRRRC | Carboxyl |
| Amine | homoC-RLVLAS | Carboxyl | Disulfide | Acetyl | YGRKKRRQRRRC | Carboxyl |
| Amine | homoC-RLVLAS | Carboxyl | Disulfide | Acetyl | YGRKKRRQRRRC | Amide |
| Amine | homoC-RLVLAS | Carboxyl | Disulfide | Amine | YGRKKRRQRRRC | Amide |
| Acetyl | homoC-RLVLAS | Amide | Disulfide | Acetyl | YGRKKRRQRRRC | Amide |

TABLE 4

HOMOCYSTEINE (homoC) - Carrier only

| | | |
|---|---|---|
| CARGO | LINKER | CARRIER |

| | | | | | | |
|---|---|---|---|---|---|---|
| N-term | Cargo | C-term | Linker | N-term | Carrier | C-term |

| | | | | | | |
|---|---|---|---|---|---|---|
| Amine | CRLVLAS | Carboxyl | Disulfide | Amine | homoC-YGRKKRRQRRR | Carboxyl |
| Acetyl | CRLVLAS | Carboxyl | Disulfide | Amine | homoC-YGRKKRRQRRR | Carboxyl |
| Acetyl | CRLVLAS | Amide | Disulfide | Amine | homoC-YGRKKRRQRRR | Carboxyl |
| Amine | CRLVLAS | Amide | Disulfide | Amine | homoC-YGRKKRRQRRR | Carboxyl |
| Amine | CRLVLAS | Carboxyl | Disulfide | Acetyl | homoC-YGRKKRRQRRR | Carboxyl |
| Amine | CRLVLAS | Carboxyl | Disulfide | Acetyl | homoC-YGRKKRRQRRR | Amide |
| Amine | CRLVLAS | Carboxyl | Disulfide | Amine | homoC-YGRKKRRQRRR | Amide |
| Acetyl | CRLVLAS | Amide | Disulfide | Acetyl | homoC-YGRKKRRQRRR | Amide |
| Amine | RLVLASC | Carboxyl | Disulfide | Amine | homoC-YGRKKRRQRRR | Carboxyl |
| Acetyl | RLVLASC | Carboxyl | Disulfide | Amine | homoC-YGRKKRRQRRR | Carboxyl |
| Acetyl | RLVLASC | Amide | Disulfide | Amine | homoC-YGRKKRRQRRR | Carboxyl |
| Amine | RLVLASC | Amide | Disulfide | Amine | homoC-YGRKKRRQRRR | Carboxyl |
| Amine | RLVLASC | Carboxyl | Disulfide | Acetyl | homoC-YGRKKRRQRRR | Carboxyl |
| Amine | RLVLASC | Carboxyl | Disulfide | Acetyl | homoC-YGRKKRRQRRR | Amide |
| Amine | RLVLASC | Carboxyl | Disulfide | Amine | homoC-YGRKKRRQRRR | Amide |
| Acetyl | RLVLASC | Amide | Disulfide | Acetyl | homoC-YGRKKRRQRRR | Amide |
| Amine | RLVLASC | Carboxyl | Disulfide | Amine | YGRKKRRQRRR-homoC | Carboxyl |
| Acetyl | RLVLASC | Carboxyl | Disulfide | Amine | YGRKKRRQRRR-homoC | Carboxyl |
| Acetyl | RLVLASC | Amide | Disulfide | Amine | YGRKKRRQRRR-homoC | Carboxyl |
| Amine | RLVLASC | Amide | Disulfide | Amine | YGRKKRRQRRR-homoC | Carboxyl |
| Amine | RLVLASC | Carboxyl | Disulfide | Acetyl | YGRKKRRQRRR-homoC | Carboxyl |
| Amine | RLVLASC | Carboxyl | Disulfide | Acetyl | YGRKKRRQRRR-homoC | Amide |
| Amine | RLVLASC | Carboxyl | Disulfide | Amine | YGRKKRRQRRR-homoC | Amide |
| Acetyl | RLVLASC | Amide | Disulfide | Acetyl | YGRKKRRQRRR-homoC | Amide |
| Amine | CRLVLAS | Carboxyl | Disulfide | Amine | YGRKKRRQRRR-homoC | Carboxyl |
| Acetyl | CRLVLAS | Carboxyl | Disulfide | Amine | YGRKKRRQRRR-homoC | Carboxyl |
| Acetyl | CRLVLAS | Amide | Disulfide | Amine | YGRKKRRQRRR-homoC | Carboxyl |
| Amine | CRLVLAS | Amide | Disulfide | Amine | YGRKKRRQRRR-homoC | Carboxyl |
| Amine | CRLVLAS | Carboxyl | Disulfide | Acetyl | YGRKKRRQRRR-homoC | Carboxyl |
| Amine | CRLVLAS | Carboxyl | Disulfide | Acetyl | YGRKKRRQRRR-homoC | Amide |
| Amine | CRLVLAS | Carboxyl | Disulfide | Amine | YGRKKRRQRRR-homoC | Amide |
| Acetyl | CRLVLAS | Amide | Disulfide | Acetyl | YGRKKRRQRRR-homoC | Amide |

TABLE 5

MERCAPTOPROPIONIC ACID (MerPC)

| | | |
|---|---|---|
| CARGO | LINKER | CARRIER |

| | | | | | | |
|---|---|---|---|---|---|---|
| N-term | Cargo | C-term | Linker | N-term | Carrier | C-term |

| | | | | | | |
|---|---|---|---|---|---|---|
| Amine | MerPC-RLVLAS | Carboxyl | Disulfide | Amine | MerPC-YGRKKRRQRRR | Carboxyl |
| | SEQ ID NO: 10 | | | | SEQ ID NO: 11 | |
| Acetyl | MerPC-RLVLAS | Carboxyl | Disulfide | Amine | MerPC-YGRKKRRQRRR | Carboxyl |
| Acetyl | MerPC-RLVLAS | Amide | Disulfide | Amine | MerPC-YGRKKRRQRRR | Carboxyl |
| Amine | MerPC-RLVLAS | Amide | Disulfide | Amine | MerPC-YGRKKRRQRRR | Carboxyl |
| Amine | MerPC-RLVLAS | Carboxyl | Disulfide | Acetyl | MerPC-YGRKKRRQRRR | Carboxyl |
| Amine | MerPC-RLVLAS | Carboxyl | Disulfide | Acetyl | MerPC-YGRKKRRQRRR | Amide |
| Amine | MerPC-RLVLAS | Carboxyl | Disulfide | Amine | MerPC-YGRKKRRQRRR | Amide |
| Acetyl | MerPC-RLVLAS | Amide | Disulfide | Acetyl | MerPC-YGRKKRRQRRR | Amide |
| Amine | MerPC-RLVLAS | Carboxyl | Disulfide | Amine | CYGRKKRRQRRR | Carboxyl |
| Acetyl | MerPC-RLVLAS | Carboxyl | Disulfide | Amine | CYGRKKRRQRRR | Carboxyl |
| Acetyl | MerPC-RLVLAS | Amide | Disulfide | Amine | CYGRKKRRQRRR | Carboxyl |
| Amine | MerPC-RLVLAS | Amide | Disulfide | Amine | CYGRKKRRQRRR | Carboxyl |
| Amine | MerPC-RLVLAS | Carboxyl | Disulfide | Acetyl | CYGRKKRRQRRR | Carboxyl |
| Amine | MerPC-RLVLAS | Carboxyl | Disulfide | Acetyl | CYGRKKRRQRRR | Amide |
| Amine | MerPC-RLVLAS | Carboxyl | Disulfide | Amine | CYGRKKRRQRRR | Amide |
| Acetyl | MerPC-RLVLAS | Amide | Disulfide | Acetyl | CYGRKKRRQRRR | Amide |
| Amine | CRLVLAS | Carboxyl | Disulfide | Amine | MerPC-YGRKKRRQRRR | Carboxyl |
| Acetyl | CRLVLAS | Carboxyl | Disulfide | Amine | MerPC-YGRKKRRQRRR | Carboxyl |
| Acetyl | CRLVLAS | Amide | Disulfide | Amine | MerPC-YGRKKRRQRRR | Carboxyl |
| Amine | CRLVLAS | Amide | Disulfide | Amine | MerPC-YGRKKRRQRRR | Carboxyl |
| Amine | CRLVLAS | Carboxyl | Disulfide | Acetyl | MerPC-YGRKKRRQRRR | Carboxyl |
| Amine | CRLVLAS | Carboxyl | Disulfide | Acetyl | MerPC-YGRKKRRQRRR | Amide |
| Amine | CRLVLAS | Carboxyl | Disulfide | Amine | MerPC-YGRKKRRQRRR | Amide |
| Acetyl | CRLVLAS | Amide | Disulfide | Acetyl | MerPC-YGRKKRRQRRR | Amide |

TABLE 6

MERCAPTOACETIC ACID (MerAC)

| | | |
|---|---|---|
| CARGO | LINKER | CARRIER |

| | | | | | | |
|---|---|---|---|---|---|---|
| N-term | Cargo | C-term | Linker | N-term | Carrier | C-term |

| | | | | | | |
|---|---|---|---|---|---|---|
| Amine | MerAC-RLVLAS | Carboxyl | Disulfide | Amine | MerAC-YGRKKRRQRRR | Carboxyl |
| Acetyl | MerAC-RLVLAS | Carboxyl | Disulfide | Amine | MerAC-YGRKKRRQRRR | Carboxyl |
| Acetyl | MerAC-RLVLAS | Amide | Disulfide | Amine | MerAC-YGRKKRRQRRR | Carboxyl |
| Amine | MerAC-RLVLAS | Amide | Disulfide | Amine | MerAC-YGRKKRRQRRR | Carboxyl |
| Amine | MerAC-RLVLAS | Carboxyl | Disulfide | Acetyl | MerAC-YGRKKRRQRRR | Carboxyl |
| Amine | MerAC-RLVLAS | Carboxyl | Disulfide | Acetyl | MerAC-YGRKKRRQRRR | Amide |
| Amine | MerAC-RLVLAS | Carboxyl | Disulfide | Amine | MerAC-YGRKKRRQRRR | Amide |
| Acetyl | MerAC-RLVLAS | Amide | Disulfide | Acetyl | MerAC-YGRKKRRQRRR | Amide |
| Amine | MerAC-RLVLAS | Carboxyl | Disulfide | Amine | CYGRKKRRQRRR | Carboxyl |
| Acetyl | MerAC-RLVLAS | Carboxyl | Disulfide | Amine | CYGRKKRRQRRR | Carboxyl |
| Acetyl | MerAC-RLVLAS | Amide | Disulfide | Amine | CYGRKKRRQRRR | Carboxyl |
| Amine | MerAC-RLVLAS | Amide | Disulfide | Amine | CYGRKKRRQRRR | Carboxyl |
| Amine | MerAC-RLVLAS | Carboxyl | Disulfide | Acetyl | CYGRKKRRQRRR | Carboxyl |
| Amine | MerAC-RLVLAS | Carboxyl | Disulfide | Acetyl | CYGRKKRRQRRR | Amide |
| Amine | MerAC-RLVLAS | Carboxyl | Disulfide | Amine | CYGRKKRRQRRR | Amide |
| Acetyl | MerAC-RLVLAS | Amide | Disulfide | Acetyl | CYGRKKRRQRRR | Amide |
| Amine | CRLVLAS | Carboxyl | Disulfide | Amine | MerAC-YGRKKRRQRRR | Carboxyl |
| Acetyl | CRLVLAS | Carboxyl | Disulfide | Amine | MerAC-YGRKKRRQRRR | Carboxyl |
| Acetyl | CRLVLAS | Amide | Disulfide | Amine | MerAC-YGRKKRRQRRR | Carboxyl |
| Amine | CRLVLAS | Amide | Disulfide | Amine | MerAC-YGRKKRRQRRR | Carboxyl |
| Amine | CRLVLAS | Carboxyl | Disulfide | Acetyl | MerAC-YGRKKRRQRRR | Carboxyl |
| Amine | CRLVLAS | Carboxyl | Disulfide | Acetyl | MerAC-YGRKKRRQRRR | Amide |
| Amine | CRLVLAS | Carboxyl | Disulfide | Amine | MerAC-YGRKKRRQRRR | Amide |
| Acetyl | CRLVLAS | Amide | Disulfide | Acetyl | MerAC-YGRKKRRQRRR | Amide |

TABLE 7

MERCAPTOBUTYRIC ACID (MerBC)

| | | |
|---|---|---|
| CARGO | LINKER | CARRIER |

| | | | | | | |
|---|---|---|---|---|---|---|
| N-term | Cargo | C-term | Linker | N-term | Carrier | C-term |

| | | | | | | |
|---|---|---|---|---|---|---|
| Amine | MerBC-RLVLAS | Carboxyl | Disulfide | Amine | MerBC-YGRKKRRQRRR | Carboxyl |
| | SEQ ID NO: 11 | | | | SEQ ID NO: 12 | |
| Acetyl | MerBC-RLVLAS | Carboxyl | Disulfide | Amine | MerBC-YGRKKRRQRRR | Carboxyl |
| Acetyl | MerBC-RLVLAS | Amide | Disulfide | Amine | MerBC-YGRKKRRQRRR | Carboxyl |
| Amine | MerBC-RLVLAS | Amide | Disulfide | Amine | MerBC-YGRKKRRQRRR | Carboxyl |
| Amine | MerBC-RLVLAS | Carboxyl | Disulfide | Acetyl | MerBC-YGRKKRRQRRR | Carboxyl |
| Amine | MerBC-RLVLAS | Carboxyl | Disulfide | Acetyl | MerBC-YGRKKRRQRRR | Amide |
| Amine | MerBC-RLVLAS | Carboxyl | Disulfide | Amine | MerBC-YGRKKRRQRRR | Amide |
| Acetyl | MerBC-RLVLAS | Amide | Disulfide | Acetyl | MerBC-YGRKKRRQRRR | Amide |
| Amine | MerBC-RLVLAS | Carboxyl | Disulfide | Amine | CYGRKKRRQRRR | Carboxyl |
| Acetyl | MerBC-RLVLAS | Carboxyl | Disulfide | Amine | CYGRKKRRQRRR | Carboxyl |
| Acetyl | MerBC-RLVLAS | Amide | Disulfide | Amine | CYGRKKRRQRRR | Carboxyl |
| Amine | MerBC-RLVLAS | Amide | Disulfide | Amine | CYGRKKRRQRRR | Carboxyl |
| Amine | MerBC-RLVLAS | Carboxyl | Disulfide | Acetyl | CYGRKKRRQRRR | Carboxyl |
| Amine | MerBC-RLVLAS | Carboxyl | Disulfide | Acetyl | CYGRKKRRQRRR | Amide |
| Amine | MerBC-RLVLAS | Carboxyl | Disulfide | Amine | CYGRKKRRQRRR | Amide |
| Acetyl | MerBC-RLVLAS | Amide | Disulfide | Acetyl | CYGRKKRRQRRR | Amide |
| Amine | CRLVLAS | Carboxyl | Disulfide | Amine | MerBC-YGRKKRRQRRR | Carboxyl |
| Acetyl | CRLVLAS | Carboxyl | Disulfide | Amine | MerBC-YGRKKRRQRRR | Carboxyl |
| Acetyl | CRLVLAS | Amide | Disulfide | Amine | MerBC-YGRKKRRQRRR | Carboxyl |
| Amine | CRLVLAS | Amide | Disulfide | Amine | MerBC-YGRKKRRQRRR | Carboxyl |
| Amine | CRLVLAS | Carboxyl | Disulfide | Acetyl | MerBC-YGRKKRRQRRR | Carboxyl |
| Amine | CRLVLAS | Carboxyl | Disulfide | Acetyl | MerBC-YGRKKRRQRRR | Amide |
| Amine | CRLVLAS | Carboxyl | Disulfide | Amine | MerBC-YGRKKRRQRRR | Amide |
| Acetyl | CRLVLAS | Amide | Disulfide | Acetyl | MerBC-YGRKKRRQRRR | Amide |

TABLE 8

Ala-Cys

| | | |
|---|---|---|
| CARGO | LINKER | CARRIER |

| | | | | | | |
|---|---|---|---|---|---|---|
| N-term | Cargo | C-term | Linker | N-term | Carrier | C-term |

| | | | | | | |
|---|---|---|---|---|---|---|
| Amine | ACRLVLAS | Carboxyl | Disulfide | Amine | ACYGRKKRRQRRR | Carboxyl |
| | SEQ ID | | | | SEQ ID NO: 14 | |
| | NO: 13 | | | | | |
| Acetyl | ACRLVLAS | Carboxyl | Disulfide | Amine | ACYGRKKRRQRRR | Carboxyl |
| Acetyl | ACRLVLAS | Amide | Disulfide | Amine | ACYGRKKRRQRRR | Carboxyl |
| Amine | ACRLVLAS | Amide | Disulfide | Amine | ACYGRKKRRQRRR | Carboxyl |
| Amine | ACRLVLAS | Carboxyl | Disulfide | Acetyl | ACYGRKKRRQRRR | Carboxyl |
| Amine | ACRLVLAS | Carboxyl | Disulfide | Acetyl | ACYGRKKRRQRRR | Amide |
| Amine | ACRLVLAS | Carboxyl | Disulfide | Amine | ACYGRKKRRQRRR | Amide |
| Acetyl | ACRLVLAS | Amide | Disulfide | Acetyl | ACYGRKKRRQRRR | Amide |
| Amine | ACRLVLAS | Carboxyl | Disulfide | Amine | CYGRKKRRQRRR | Carboxyl |
| Acetyl | ACRLVLAS | Carboxyl | Disulfide | Amine | CYGRKKRRQRRR | Carboxyl |
| Acetyl | ACRLVLAS | Amide | Disulfide | Amine | CYGRKKRRQRRR | Carboxyl |
| Amine | ACRLVLAS | Amide | Disulfide | Amine | CYGRKKRRQRRR | Carboxyl |
| Amine | ACRLVLAS | Carboxyl | Disulfide | Acetyl | CYGRKKRRQRRR | Carboxyl |
| Amine | ACRLVLAS | Carboxyl | Disulfide | Acetyl | CYGRKKRRQRRR | Amide |
| Amine | ACRLVLAS | Carboxyl | Disulfide | Amine | CYGRKKRRQRRR | Amide |
| Acetyl | ACRLVLAS | Amide | Disulfide | Acetyl | CYGRKKRRQRRR | Amide |
| Amine | CRLVLAS | Carboxyl | Disulfide | Amine | ACYGRKKRRQRRR | Carboxyl |
| Acetyl | CRLVLAS | Carboxyl | Disulfide | Amine | ACYGRKKRRQRRR | Carboxyl |
| Acetyl | CRLVLAS | Amide | Disulfide | Amine | ACYGRKKRRQRRR | Carboxyl |
| Amine | CRLVLAS | Amide | Disulfide | Amine | ACYGRKKRRQRRR | Carboxyl |
| Amine | CRLVLAS | Carboxyl | Disulfide | Acetyl | ACYGRKKRRQRRR | Carboxyl |
| Amine | CRLVLAS | Carboxyl | Disulfide | Acetyl | ACYGRKKRRQRRR | Amide |
| Amine | CRLVLAS | Carboxyl | Disulfide | Amine | ACYGRKKRRQRRR | Amide |
| Acetyl | CRLVLAS | Amide | Disulfide | Acetyl | ACYGRKKRRQRRR | Amide |

TABLE 9
DIMER

| CARGO | | | LINKER | CARRIER | | |
|---|---|---|---|---|---|---|
| N-term | Cargo | C-term | Linker | N-term | Carrier | C-term |
| Amine | SEQ IDNO:15 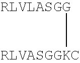 SEQ IDNO:16 | Carboxyl | Disulfide | Amine | CYGRKKRRQRRR | Carboxyl |
| Acetyl | 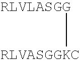 | Carboxyl | Disulfide | Amine | CYGRKKRRQRRR | Carboxyl |
| Acetyl | 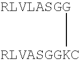 | Amide | Disulfide | Amine | CYGRKKRRQRRR | Carboxyl |
| Amine | 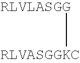 | Amide | Disulfide | Amine | CYGRKKRRQRRR | Carboxyl |
| Amine | 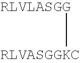 | Carboxyl | Disulfide | Acetyl | CYGRKKRRQRRR | Carboxyl |
| Amine | 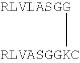 | Carboxyl | Disulfide | Acetyl | CYGRKKRRQRRR | Amide |
| Amine | 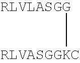 | Carboxyl | Disulfide | Amide | CYGRKKRRQRRR | Amide |
| Acetyl | 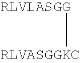 | Amide | Disulfide | Acetyl | CYGRKKRRQRRR | Amide |
| Amide | 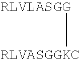 | Carboxyl | Disulfide | Amide | YGRKKRRQRRRC | Carboxyl |
| Acetyl | 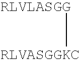 | Carboxyl | Disulfide | Amide | YGRKKRRQRRRC | Carboxyl |
| Acetyl | 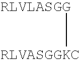 | Amide | Disulfide | Amide | YGRKKRRQRRRC | Carboxyl |
| Amide | 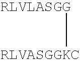 | Amide | Disulfide | Amide | YGRKKRRQRRRC | Carboxyl |
| Amide | 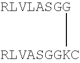 | Carboxyl | Disulfide | Acetyl | TGRKKRRQRRRC | Carboxyl |
| Amine | 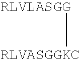 | Carboxyl | Disulfide | Acetyl | YGRKKRRQRRRC | Amide |
| Amine | 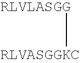 | Carboxyl | Disulfide | Amide | YGRKKRRQRRRC | Amide |
| Acetyl | 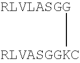 | Amide | Disulfide | Acetyl | YGRKKRRQRRRC | Amide |

TABLE 10
DIMER-HOMORCYSTEINE (Cargo)

| CARGO | | | LINKER | CARRIER | | |
|---|---|---|---|---|---|---|
| N-term | Cargo | C-term | Linker | N-term | Carrier | C-term |
| Amine | 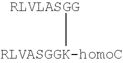 SEQ ID NO:17 | Carboxyl | Disulfide | Amine | CYGRKKRRQRRR | Carboxyl |
| Acetyl | 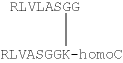 | Carboxyl | Disulfide | Amine | CYGRKKRRQRRR | Carboxyl |
| Acetyl | 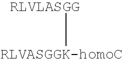 | Amide | Disulfide | Amine | CYGRKKRRQRRR | Carboxyl |
| Amine | 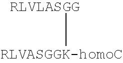 | Amide | Disulfide | Amine | CYGRKKRRQRRR | Carboxyl |
| Amine | 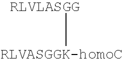 | Carboxyl | Disulfide | Acetyl | CYGRKKRRQRRR | Carboxyl |
| Amine | 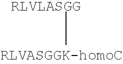 | Carboxyl | Disulfide | Acetyl | CYGRKKRRQRRR | Amide |
| Amine | 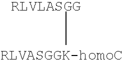 | Carboxyl | Disulfide | Amine | CYGRKKRRQRRR | Amide |
| Acetyl | 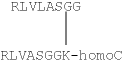 | Amide | Disulfide | Acetyl | CYGRKKRRQRRR | Amide |
| Amine | 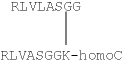 | Carboxyl | Disulfide | Amine | YGRKKRRQRRRC | Carboxyl |
| Acetyl | 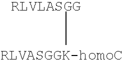 | Carboxyl | Disulfide | Amine | YGRKKRRQRRRC | Carboxyl |
| Acetyl | 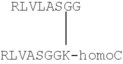 | Amide | Disulfide | Amine | YGRKKRRQRRRC | Carboxyl |
| Amine | 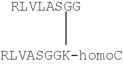 | Amide | Disulfide | Amine | YGRKKRRQRRRC | Carboxyl |
| Amine | 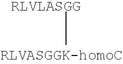 | Carboxyl | Disulfide | Acetyl | YGRKKRRQRRRC | Carboxyl |
| Amine | 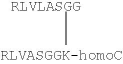 | Carboxyl | Disulfide | Acetyl | YGRKKRRQRRRC | Amide |
| Amine | 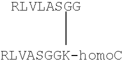 | Carboxyl | Disulfide | Amine | YGRKKRRQRRRC | Amide |
| Acetyl | 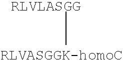 | Amide | Disulfide | Acetyl | YGRKKRRQRRRC | Amide |

TABLE 11
DIMER-HOMOCYSTEINE (Carrier)

| CARGO | | | LINKER | CARRIER | | |
|---|---|---|---|---|---|---|
| N-term | Cargo | C-term | Linker | N-term | Carrier | C-term |
| Amine | 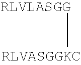 | Carboxyl | Disulfide | Amine | homoC-YGRKKRRQRRR | Carboxyl |
| Acetyl | 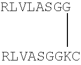 | Carboxyl | Disulfide | Amine | homoC-YGRKKRRQRRR | Carboxyl |
| Acetyl | 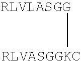 | Amide | Disulfide | Amine | homoC-YGRKKRRQRRR | Carboxyl |
| Amine | 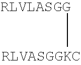 | Amide | Disulfide | Amine | homoC-YGRKKRRQRRR | Carboxyl |
| Amine | 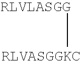 | Carboxyl | Disulfide | Acetyl | homoC-YGRKKRRQRRR | Carboxyl |
| Amine | 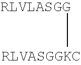 | Carboxyl | Disulfide | Acetyl | homoC-YGRKKRRQRRR | Amide |
| Amine | 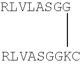 | Carboxyl | Disulfide | Amine | homoC-YGRKKRRQRRR | Amide |
| Acetyl | 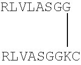 | Amide | Disulfide | Acetyl | homoC-YGRKKRRQRRR | Amide |
| Amine | 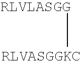 | Carboxyl | Disulfide | Amine | YGRKKRRQRRR-homoC | Carboxyl |
| Acetyl | 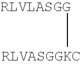 | Carboxyl | Disulfide | Amine | YGRKKRRQRRR-homoC | Carboxyl |
| Acetyl | 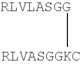 | Ammide | Disulfide | Amine | YGRKKRRQRRR-homoC | Carboxyl |
| Amine | 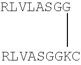 | Amide | Disulfide | Amine | YGRKKRRQRRR-homoC | Carboxyl |
| Amine | 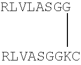 | Carboxyl | Disulfide | Acetyl | YGRKKRRQRRR-homoC | Carboxyl |
| Amine | 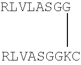 | Carboxyl | Disulfide | Acetyl | YGRKKRRQRRR-homoC | Ammide |
| Amine | 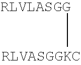 | Carboxyl | Disulfide | Amine | YGRKKRRQRRR-homoC | Amide |
| Acetyl | 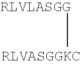 | Amide | Disulfide | Acetyl | YGRKKRRQRRR-homoC | Amide |

TABLE 12
DIMER-HOMOCYSTEINE (Both)

| CARGO | | | LINKER | CARRIER | | |
|---|---|---|---|---|---|---|
| N-term | Cargo | C-term | Linker | N-term | Carrier | Carrier |
| Amine | 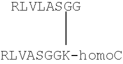 | Carboxyl | Disulfide | Amine | homoC-YGRKKRRQRRR | Carboxyl |
| Acetyl | 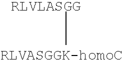 | Carboxyl | Disulfide | Amine | homoC-YGRKKRRQRRR | Carboxyl |
| Acetyl | 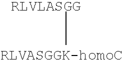 | Amide | Disulfide | Amine | homoC-YGRKKRRQRRR | Carboxyl |
| Amine | 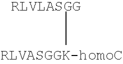 | Amide | Disulfide | Amine | homoC-YGRKKRRQRRR | Carboxyl |
| Amine | 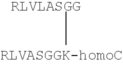 | Carboxyl | Disulfide | Acetyl | homoC-YGRKKRRQRRR | Carboxyl |
| Amine | 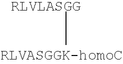 | Carboxyl | Disulfide | Acetyl | homoC-YGRKKRRQRRR | Amide |
| Amine | 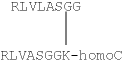 | Carboxyl | Disulfide | Amine | homoC-YGRKKRRQRRR | Amide |
| Acetyl | 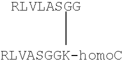 | Amide | Disulfide | Acetyl | homoC-YGRKKRRQRRR | Amide |
| Amine | 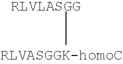 | Carboxyl | Disulfide | Amine | YGRKKRRQRRR-homoC | Carboxyl |
| Acetyl | 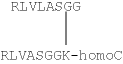 | Carboxyl | Disulfide | Amine | YGRKKRRQRRR-homoC | Carboxyl |
| Acetyl | 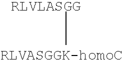 | Amide | Disulfide | Amine | YGRKKRRQRRR-homoC | Carboxyl |
| Amine | 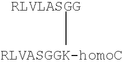 | Amide | Disulfide | Amine | YGRKKRRQRRR-homoC | Carboxyl |
| Amine | 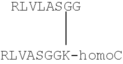 | Carboxyl | Disulfide | Acetyl | YGRKKRRQRRR-homoC | Carboxyl |
| Amine | 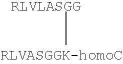 | Carboxyl | Disulfide | Acetyl | YGRKKRRQRRR-homoC | Amide |
| Amine | 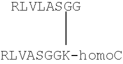 | Carboxyl | Disulfide | Amine | YGRKKRRQRRR-homoC | Amide |
| Acetyl | 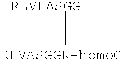 | Amide | Disulfide | Acetyl | YGRKKRRQRRR-homoC | Amide |

TABLE 13
DIMER-MERCAPTOPROPIONIC ACID

| | | |
|---|---|---|
| CARGO | LINKER | CARRIER |

| N-term | Cargo | C-term | Linker | N-term | Carrier | C-term |
|---|---|---|---|---|---|---|
| Amine | 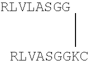 | Carboxyl | Disulfide | Amine | MerPC-YGRKKRRQRRR | Carboxyl |
| Acetyl | 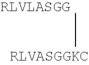 | Carboxyl | Disulfide | Amine | MerPC-YGRKKRRQRRR | Carboxyl |
| Acetyl | 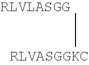 | Amide | Disulfide | Amine | MerPC-YGRKKRRQRRR | Carboxyl |
| Amine | 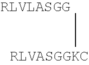 | Ammide | Disulfide | Amine | MerPC-YGRKKRRQRRR | Carboxyl |
| Amine | 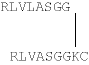 | Carboxyl | Disulfide | Acetyl | MerPC-YGRKKRRQRRR | Carboxyl |
| Amine | 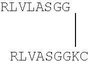 | Carboxyl | Disulfide | Acetyl | MerPC-YGRKKRRQRRR | Amide |
| Amine | 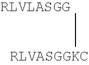 | Carboxyl | Disulfide | Amine | MerPC-YGRKKRRQRRR | Amide |
| Acetyl | 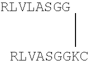 | Amide | Disulfide | Acetyl | MerPC-YGRKKRRQRRR | Amide |

TABLE 14
DIMER-MERCAPTOACETIC ACID

| CARGO | | | LINKER | CARRIER | | |
|---|---|---|---|---|---|---|
| N-term | Cargo | C-term | Linker | N-term | Carrier | C-term |
| Amine | 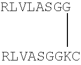 | Carboxyl | Disulfide | Amine | MerAC-YGRKKRRQRRR | Carboxyl |
| Acetyl | 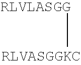 | Carboxyl | Disulfide | Amine | MerAC-YGRKKRRQRRR | Carboxyl |
| Acetyl | 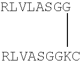 | Amide | Disulfide | Amine | MerAC-YGRKKRRQRRR | Carboxyl |
| Amine | 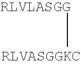 | Amide | Disulfide | Amine | MerAC-YGRKKRRQRRR | Carboxyl |
| Amine | 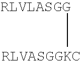 | Carboxyl | Disulfide | Acetyl | MerAC-YGRKKRRQRRR | Carboxyl |
| Amine | 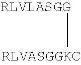 | Carboxyl | Disulfide | Acetyl | MerAC-YGRKKRRQRRR | Amide |
| Amine | 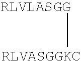 | Carboxyl | Disulfide | Amine | MerAC-YGRKKRRQRRR | Amide |
| Acetyl | 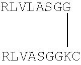 | Amide | Disulfide | Acetyl | MerAC-YGRKKRRQRRR | Amide |

TABLE 15
DIMER-MERCAPTOBUTYRIC ACID

| CARGO | | | LINKER | CARRIER | | |
|---|---|---|---|---|---|---|
| N-term | Cargo | C-term | Linker | N-term | Carrier | C-term |
| Amine | 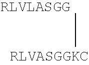 | Carboxyl | Disulfide | Amine | MerBC-YGRKKRRQRRR | Carboxyl |
| Acetyl | 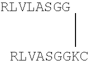 | Carboxyl | Disulfide | Amine | MerBC-YGRKKRRQRRR | Carboxyl |
| Acetyl | 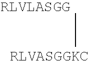 | Amide | Disulfide | Amine | MerBC-YGRKKRRQRRR | Carboxyl |
| Amine | 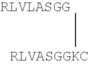 | Amide | Disulfide | Amine | MerBC-YGRKKRRQRRR | Carboxyl |
| Amine | 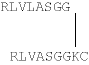 | Carboxyl | Disulfide | Acetyl | MerBC-YGRKKRRQRRR | Carboxyl |
| Amine | 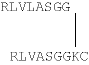 | Carboxyl | Disulfide | Acetyl | MerBC-YGRKKRRQRRR | Amide |
| Amine | 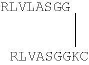 | Carboxyl | Disulfide | Amine | MerBC-YGRKKRRQRRR | Amide |
| Acetyl | 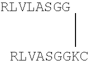 | Amide | Disulfide | Acetyl | MerBC-YGRKKRRQRRR | Amide |

TABLE 16
LINEAR

| N-term | Sequence | C-term |
|---|---|---|
| Amine | RLVLASGGYGRKKRRQRRR | Carboxyl |
| Acetyl | RLVLASGGYGRKKRRQRRR | Carboxyl |
| | SEQ ID NO:18 | |
| Amine | RLVLASGGYGRKKRRQRRR | Amide |
| Acetyl | RLVLASGGYGRKKRRQRRR | Amide |
| Amine | YGRKKRRQRRRGGRLVAS | Carboxyl |
| | SEQ ID NO:19 | |
| Acetyl | YGRKKRRQRRRGGRLVLAS | Carboxyl |
| Amine | YGRKKRRQRRRGGRLVLAS | Amide |
| Acetyl | YGRKKRRQRRRGGRLVLAS | Amide |
| Amine | 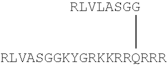 SEQ ID NO:19 | Carboxyl |
| Acetyl | 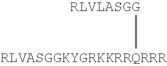 | Carboxyl |
| Amine | 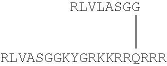 | Amide |
| Acetyl | 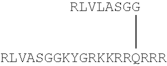 | Amide |
| Amine | 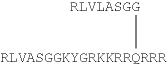 | Carboxyl |
| Acetyl | 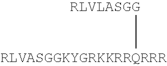 | Carboxyl |
| Amine | 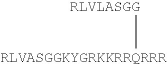 | Amide |
| Acetyl | 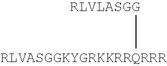 | Amide |

TABLE 17

LINEAR-retroinverso (lower case)

| | | |
|---|---|---|
| N-term | Sequence | C-term |

| | | |
|---|---|---|
| Amine | salvlrGGrrrqrrkkrgy | Carboxyl |
| | SEQ ID NO: 20 | |
| Acetyl | salvlrGGrrrqnkkrgy | Carboxyl |
| Amine | salvlrGGrrrqrrkkrgy | Amide |
| Acetyl | salvlrGGrrrqrrkkrgy | Amide |
| Amine | salvlrGGYGRKKRRQRRR | Carboxyl |
| | SEQ ID NO: 21 | |
| Acetyl | salvlrGGYGRKKRRQRRR | Carboxyl |
| Amine | salvlrGGYGRKKRRQRRR | Amide |
| Acetyl | salvlrGGYGRKKRRQRRR | Amide |

Additional variables:
All combinations and permutations of homocystiene with mercapto[proprionic, acetic, butyric]acid are applicable
   Homocysteine cargos with any mercapto[proprionic, acetic, butyric]acid carrier and vice versa
   Homocysteine can be N-term or C-term in all cases
   mercapto[proprionic, acetic, butyric]acids can only be N-term
Carrier in all tables above can be replaced with Antennapedia, poly arginine or other carriers

As discussed more fully below, it is preferable that the γPKC inhibitory peptide be chemically associated with a carrier moiety, such as a carrier peptide. In one embodiment, the inhibitory peptide and the carrier peptide are linked via a disulfide bond. Electrostatic and hydrophobic interactions can also be exploited to associate chemically the carrier moiety with the γPKC inhibitory peptide. In the case of the forming a disulfide bond, it may be advantageous to add a Cys residue to the PKC inhibitory peptide sequence or to the carrier peptide sequence. The Cys residue can be added to the amino or carboxy termini, or both. The Cys residue can also be located within the amino acid sequence of the cargo or carrier peptides. Such endogenous Cys residues have been shown to stabilize a disulfide bond linkage between the carrier and cargo peptides.

Carrier Moiety

A wide variety of molecules (particularly macromolecules such as peptides) intended for cellular uptake have been found to be poorly transported across cell membranes. Among the solutions proposed to facilitate cellular uptake have been the use of carrier moieties such as cationic (i.e., positively charged) polymers, peptides and antibody sequences, including polylysine, polyarginine, Antennapedia-derived peptides, HIV Tat-derived peptides and the like. (See, for example, U.S. Pat. Nos. and Publications Nos. 4,847,240, 5,888,762, 5,747,641, 6,593,292, US2003/0104622, US2003/0199677 and US2003/0206900.)

Methods of Use and Formulations

The modified peptides described herein are useful for the prevention and treatment of pain. For the purposes of this discussion, pain, and the treatment thereof, is categorized into different classes: treatment of acute, chronic, neuropathic, and inflammatory pain. The modified γPKC inhibitory peptides described herein are useful for the treatment of acute, chronic, neuropathic, and inflammatory pain.

Interestingly, the compounds disclosed herein are also useful in attenuated or preventing the development of neuropathic pain caused by a plurality of stimuli. The present disclosure contemplates that the administration of the γPKC inhibitory peptides described herein, either prophylactically, with at the same time as a pain inducing stimulus, or subsequent to receiving the pain inducing stimulus will be effective to attenuate or prevent the development of the chronic inflammatory or neuropathic pain condition.

Once a cargo/carrier peptide construct has been assembled and tested for increased stability, potency, or both as compared to a prototype, the construct is placed into a pharmaceutically acceptable formulation for administration to a subject prior to, during, or continuously through a pain inducing event.

A “pharmaceutically acceptable formulation” comprises one that is suitable for administering the modified γPKC inhibitor in a manner that gives the desired results and does not also produce adverse side effects sufficient to convince a physician that the potential harm to a patient is greater than the potential benefit to that patient. The components of a suitable pharmaceutically acceptable formulation for use with a modified γPKC inhibitors are determined in part by the route and method of administration. The formulations generally comprise one or more modified γPKC inhibitory peptides incorporated into a pharmaceutically acceptable carrier typically comprising simple chemicals such as sugars, amino acids or electrolytes. Exemplary solutions are typically prepared with saline or buffer. The pharmaceutically acceptable carrier may contain excipients which are well known in the art, and may be used in a variety of formulations. See, e.g., Remington's Pharmaceutical Sciences, 18th Edition, A. R. Gennaro, Editor, Mack Publishing Company (1990); Remington: The Science and Practice of Pharmacy, 20th Edition, A. R. Gennaro, Editor, Lippincott Williams & Wilkins (2000); Handbook of Pharmaceutical Excipients, 3rd Edition, A. H. Kibbe, Editor, American Pharmaceutical Association, and Pharmaceutical Press (2000); and Handbook of Pharmaceutical Additives, compiled by Michael and Irene Ash, Gower (1995).

Inhibitor dosage in the formulation will vary according to a variety of parameters influenced by the stability and potency of the cargo/carrier construct, the route of administration, and desired dosing regime. Daily dosages in the range of 1 μg/kg-100 mg/kg of body weight, preferably 1 μg/kg-1 mg/kg and most preferably 10 μg/kg-1 mg/kg are contemplated.

Modified γPKC inhibitors can be administered locally or systemically. Local administration can be achieved by topical administration, intradermal administration, intrathecal administration, intraperitoneal administration, or subcutaneous injection. Systemic administration of a modified γPKC inhibitor is preferably parenteral, although oral, buccal, and intranasal administration is also contemplated. Parenteral administration is generally characterized by injection, either subcutaneously, intramuscularly, intraperitoneal, and intravenously. Injectable forms of the modified inhibitory peptides can be prepared in conventional forms, either as liquid solutions or suspensions, solid (e.g., dried or lyophilized) forms suitable for reconstitution into solution or suspension in liquid prior to injection, or as emulsions. Generally, suitable excipients include, for example, water, saline, dextrose, glycerol, ethanol or the like. In addition, minor amounts of non-toxic auxiliary substances can be employed, such as wetting or emulsifying agents, pH buffering agents, solubility enhancers, tonicifiers and the like including, for example, sodium acetate, sorbitan monolaurate, triethanolamine oleate, cyclodextrins, etc.

The modified γPKC inhibitory peptides can be administered to treat pain as necessary. For prophylaxis, the modified γPKC compound may be administered prior to a pain-inducing event. For example, the peptide can be administered 5, 10, 15, 20, 25, 30, 35, 40, 45, 50, 55 minutes, one hour, several hours, one day, several days, one week, or weeks prior ahead of an anticipated pain-inducing event. Even longer periods of prophylactic administration can be achieved using modified peptides that are particularly stable in vivo, or by using a sustained release formulation of the peptide, e.g. delivery by intrathecal pump.

### EXAMPLES

The following examples serve to describe more fully the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### Example 1

### Administration of γPKC Inhibitor Reduces Membrane Bound Enzyme

Male Holtzman rats (Harlan, Indianapolis, Ind.) were used in the studies discussed below. Efforts were made throughout the experiment to minimize animal discomfort and to reduce the number of animals used. All rats (200-250 g at time of nerve transection) were housed in a 12-hour light/dark cycle (7 AM lights turned on) with food and water available ad libitum.

L5 spinal nerve transection were performed on the study animals. Rats were anesthetized with halothane in O₂ carrier (induction 4%, maintenance 2%). A small incision to the skin overlaying L5-S1 was made followed by retraction of the paravertebral musculature from the vertebral transverse processes. The L6 transverse process was partially removed exposing the L4 and L5 spinal nerves. The L5 spinal nerve was identified, lifted slightly, and transected. The wound was irrigated with saline and closed in two layers with 3-0 polyester suture (fascial plane) and surgical skin staples.

Western blot analysis was performed on lumbar spinal cord samples taken from animals seven (7) days post-transection. The animals were treated with a γPKC inhibit administered using a subcutaneous pump providing the inhibitor at 10, 100, or 1000 pmoles.

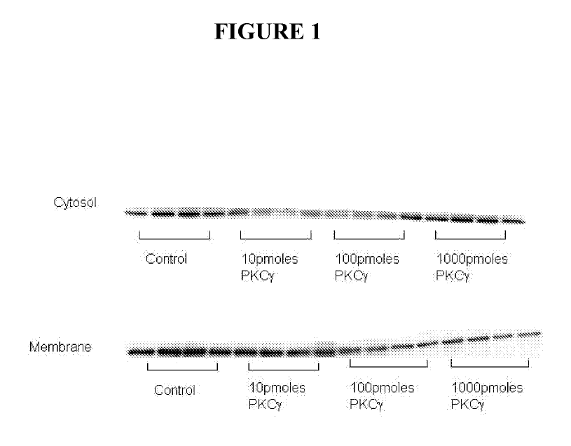

As shown in FIG. 1, increasing amounts of the inhibitor resulted in decreased amounts of detectable γPKC in the membrane preparations. Increased levels of the enzyme were detected in the cytosolic samples tested. These results demonstrate that subcutaneous administration of γPKC inhibitor peptides were effective to induce translocation of the γPKC enzyme.

### Example 2

### Prevention of Peripheral Nerve Injury-Induced Mechanical Allodynia with a Modified γPKC Inhibitory Peptide

Using a systemic preventative paradigm, a modified γPKC inhibitory peptide treatment was initiated just prior to surgery, by the implantation of a subcutaneous infusion pump. Infusion was continued for 7 days.

As previously described in Sweitzer et al., (1999) Brain Res 829: 209-221, all animals were tested for mechanical allodynia with 2- and 12-g von Frey filaments (Stoelting, Wood Dale, Ill.) on the ipsilateral hindpaw. Animals were acclimated to the testing procedure. Three baseline measurements were collected before the day of surgery. Rats were subjected to three sets of 10 stimulations with each filament with at least 10 min between each set of stimulations to prevent sensitization. Allodynia was characterized as an intense withdrawal of the paw to this normally non-noxious stimulus. Results are reported as the average number of paw withdrawals out of 30 stimulations with either the 2- or 12-g von Frey filament.

A crossover study (n=8/treatment) with sc infusion pump placement was also completed. One group of animals was treated with a preventative pain paradigm in which treatment was initiated upon L5 spinal nerve transection and continued to day 7 post-transection. At day 7 post-transection PKC inhibitor treatment was terminated and the animals were followed out to day 14. A second group of animals, in an existing pain paradigm, received a sc pump on day 7 post-transection and continued to day 14.

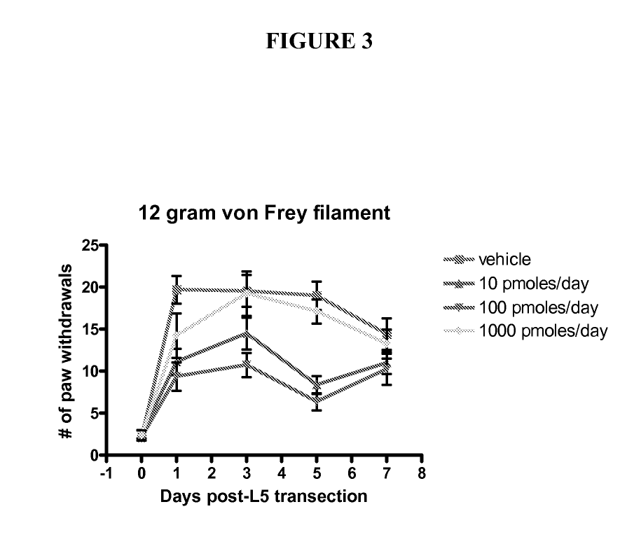

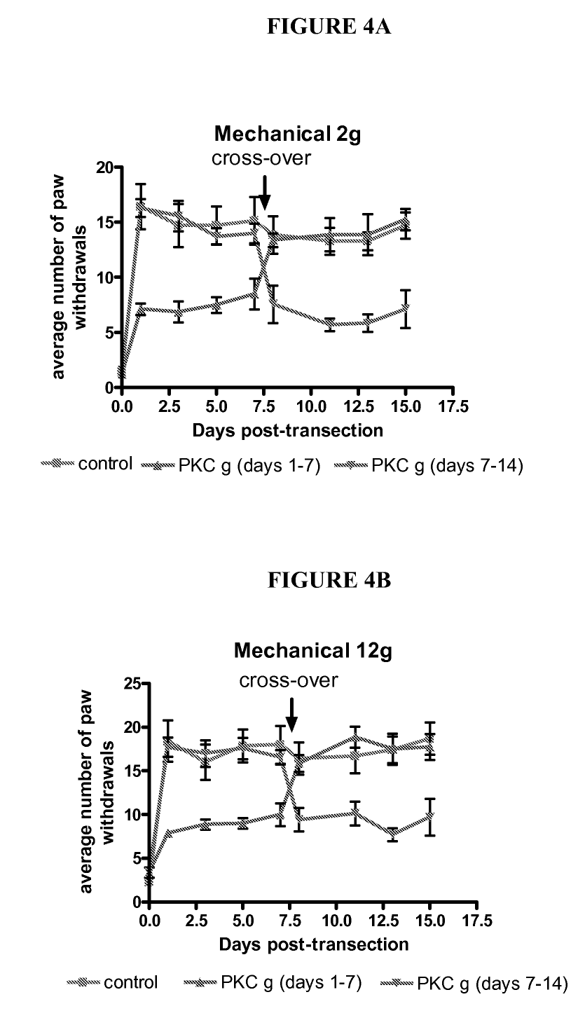

As shown in FIGS. 2, 3, and 4 (crossover study), administration of 10 and 100 pmoles of the γPKC inhibitor peptide was effective to reduce mechanical allodynia response to 2 and 12 gram von Frey filaments. Interestingly, a higher dose is not anti-allodynic. This result is similar to results produced from work using a εPKC epsilon inhibitor, although here concentrations of the gamma inhibitor were 10× higher than those used the εPKC inhibitor.

### Example 3

### Attenuation of Thermal Hyperalgesia with a Modified γPKC Inhibitory Peptide

A radiant heat source was focused onto the plantar surface of the paw of freely-moving animals housed in an acrylic testing chambers (4″×8″×4″) and paw withdrawal latency was measured to evaluate the impact of modified a γPKC inhibitory peptide on thermal hyperalgesia. Pilot experiments were conducted to determine the lamp intensity required to provide a paw flick latency of ˜10 sec in untreated animals. To ensure that no tissue damage occurs, all tests had a 30 second cutoff, according to the manufacturer's specification. Prior to inflammatory stimulation, both paws of each animal were tested for baseline sensitivity. Each test consisted of 3 measurements of same paw, with a minimum 5 minute interval between each determination. The paw withdrawal threshold was the average of these three determinations.

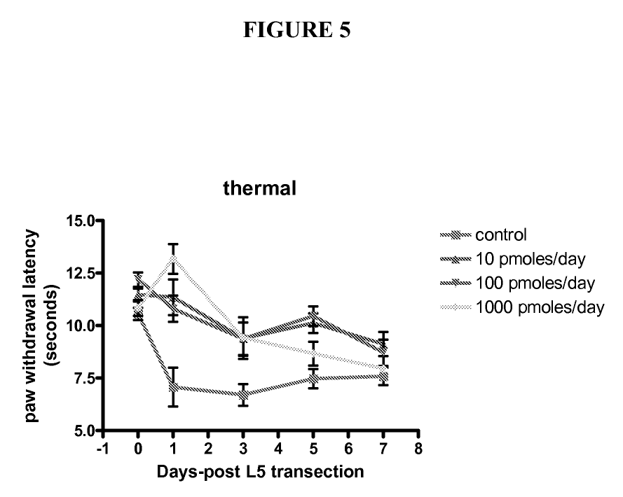

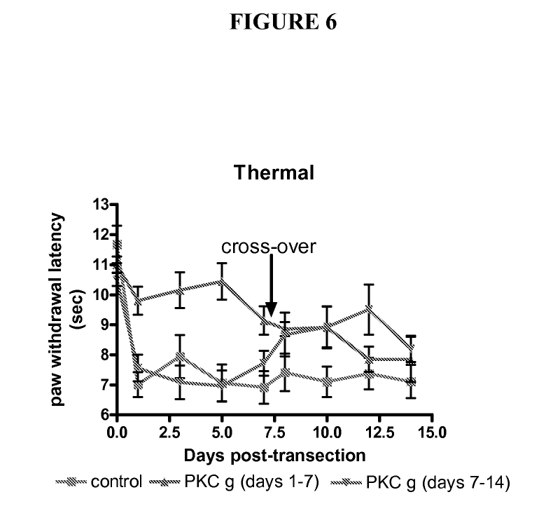

As shown in FIGS. 5 and 6, administration of the γPKC inhibitory peptide was an effective anti-hyperalgesic agent until day 7. The data in FIG. 6 shows the results of the crossover study, performed as described in Example 1.

### Example 4

### Subcutaneous Challenge Using a Modified γPKC Inhibitory Peptide

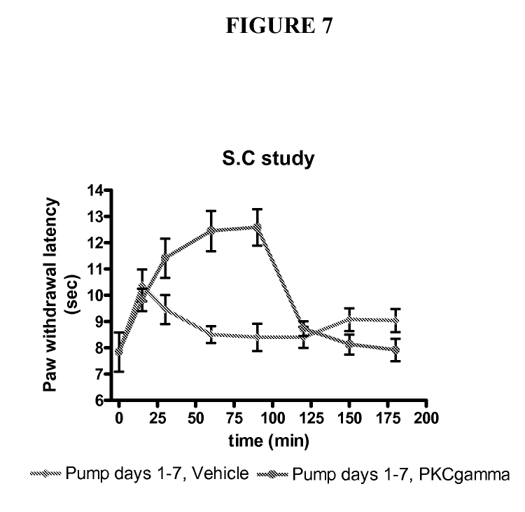

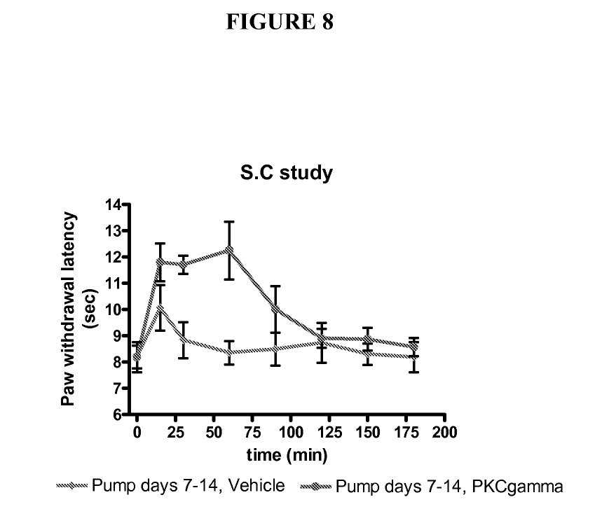

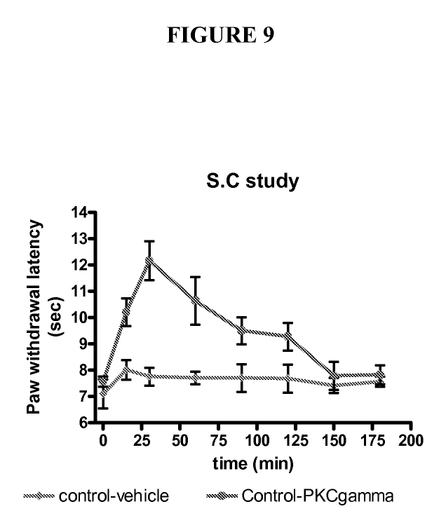

A study to evaluate the effectiveness of subcutaneous administration of modified γPKC inhibitory peptides. Animals were prepared in accordance with the methods described in Example 2. One group of animals were administered a γPKC inhibitory peptide for days 1-7 post-transection prior to challenge. The second group was administered a γPKC inhibitory peptide for days 7-14 post-transection prior to challenge. The third group was challenged without prior administration of an inhibitory peptide. In all three groups the animals received a subcutaneous challenge of 100 pmoles of the inhibitory peptide or vehicle, which was administered on day 14 post-transection. Paw withdrawal latency was measured then measured. The data from the first group, second, and third groups is shown in FIGS. 7, 8, and 9, respectively. A number of results from these studies are particularly interesting. First, paw withdrawal latency remained elevated over base line from more than 100 minutes in all groups receiving the inhibitory peptide, regardless of prior inhibitory peptide administration. Second, even animals that received no prior treatment with the peptide showed a significant decrease in paw withdrawal latency as compared to vehicle control. Third, the protective effect of the inhibitory peptide administered subcutaneously was systemic, that is applied to all four paws, and not local.

Alternative embodiments will become apparent to those skilled in the art to which the present invention pertains without departing from its spirit and scope. Accordingly, the scope of the present invention is defined by the appended claims rather than the foregoing description.

1. An gamma protein kinase C (γPKC) inhibitory peptide composition, comprising:
an γPKC inhibitory peptide covalently linked to an intracellular carrier peptide, wherein the intracellular carrier peptide, the inhibitory peptide, or both are modified at the N-terminus.

2. The composition of claim 1, wherein the PKC inhibitory peptide is linked to the intracellular carrier peptide by a disulfide bond.

3. The composition of claim 1, wherein the intracellular carrier peptide is a modified tat peptide comprising YGRKKRRQRRR (SEQ ID NO:26).

4. The composition of claim 1, wherein the intracellular carrier peptide is a modified tat peptide comprising CYGRKKRRQRRR (SEQ ID NO:4).

5. The composition of claim 3, wherein the modified tat peptide is substituted at its N-terminal end by an acyl, alkyl, or sulfonyl group.

6. The composition of claim 5, wherein the modified tat peptide is acylated at its N-terminal end.

7. The composition of claim 1, wherein the tat peptide is further modified at its C-terminal end.

8. The composition of claim 1, wherein the inhibitory peptide comprises the amino acid sequence of R-L-V-L-A-S (SEQ ID NO:1) and a terminal Cys.

9. The composition of claim 8, wherein the terminal Cys is located at the C-terminus of the inhibitory peptide.

10. The composition of claim 1, wherein the inhibitory peptide comprises the amino acid sequence of R-L-V-L-A-S-G-G (SEQ ID NO:16) and a terminal Cys.

11. The composition of claim 10, wherein the terminal Cys is located at the C-terminus of the inhibitory peptide.

12. The composition of claim 1, wherein the tat peptide is further modified by formation of an amide at its C-terminal end.

13. The composition of claim 1, wherein the PKC inhibitory peptide is covalently linked to a side chain of an amino acid of the modified tat peptide.

14. The composition of claim 13, wherein the PKC inhibitory peptide is covalently linked to a side chain of a residue selected from cysteine, glutamic acid, aspartic acid, serine, threonine, lysine, tyrosine and glutamine.

15. The composition of claim 13, wherein the PKC inhibitory peptide is covalently linked to a side chain of the N-terminal cysteine residue.

16. The composition of claim 15, wherein the N-terminal cysteine of the tat peptide is acylated.

17. The composition of claim 15, wherein the C-terminal arginine of the tat peptide is a primary carboxamide.

18. The composition of claim 15, wherein the PKC inhibitory peptide is modified by either acylation at its N-terminal end, or amidation at its C-terminal end, or by both acylation at its N-terminal end and amidation at its C-terminal end.

19. The composition of claim 1, wherein the in tat peptide is Ac—YGRKKRRQRRRC—NH₂ (SEQ ID NO:5).

20. The composition of claim 19, wherein the PKC inhibitory peptide is covalently linked to the tat peptide through the sulfhydryl group of the cysteine residue of the tat peptide.

21. The composition of claim 1, which further comprises a second membrane transport peptide.

22. A linear therapeutic peptide, comprising:
a carrier peptide and a γPKC inhibitory cargo peptide, wherein the carrier peptide and the cargo peptide are linked by a peptide bond.

23. The linear therapeutic peptide of claim 22, further comprising a linker peptide positioned between the carrier peptide and the cargo peptide, wherein the carrier peptide and the cargo peptide are linked to the linker peptide by a peptide bond.

24. A method of treating pain, comprising:
administering an effective amount of a modified gamma protein kinase C (γPKC) inhibitory construct to a subject suffering from pain, wherein the modified γPKC peptide is more stable, more potent, or both as compared to a prototype sequence.

25. The method of claim 24, wherein the pain suffered by the subject is selected from the group consisting of acute pain, chronic pain, neuropathic pain, and inflammatory pain.

26. The method of claim 24, wherein increased potency results from a faster onset of action or a longer duration of activity relative to the prototype sequence.

27. The method of claim 24, wherein the modified γPKC inhibitory peptide is administered to the subject prior to, during, or subsequent to the subject receiving a pain stimulus.

28. The method of claim 27, wherein the inhibitory peptide is administered 5, 10, 15, 20, 25, 30, 35, 40, 45, 50, 55, one hour, several hours, one day, several days, one week, or weeks prior to the pain stimulus.
